(19) United States
(12) Patent Application Publication
(10) Pub. No.: US 2011/0288125 A1
(43) Pub. Date: Nov. 24, 2011

(54) NOVEL USE OF PIPERINE

(75) Inventor: Tae Sun Park, Seoul (KR)

(73) Assignee: Industry-Academic Cooperation Foundation, Yonsei University, Seoul (KR)

(21) Appl. No.: 13/194,582

(22) Filed: Jul. 29, 2011

Related U.S. Application Data
(63) Continuation of application No. PCT/KR2009/006207, filed on Oct. 27, 2009.

(30) Foreign Application Priority Data
Feb. 2, 2009 (KR) 10-2009-00008026

(51) Int. Cl. A61K 31/4525; A61P 3/04; C07D 405/10

(52) U.S. Cl. 514/321; 546/197

(57) ABSTRACT

The present invention is related to novel use of piperine, and more specifically, it is related to a pharmaceutical or food composition for preventing and treating obesity comprising piperine or a salt thereof as an active ingredient. The present invention is related to novel use of piperine, and it provides a pharmaceutical or food composition for preventing and treating obesity comprising piperine or a salt thereof as an active ingredient. The composition comprising piperine of the present disclosure is effective on suppression of abdominal fat accumulation and body weight loss. Therefore, the composition can be effectively used to prevent and treat obesity.

### CROSS REFERENCE TO RELATED APPLICATIONS

This application is a continuation of PCT/KR2009/006207 filed on Oct. 27, 2009 which claims priority to Korean Patent Application No. 10-2009-0008026 filed on Feb. 2, 2009, the entire contents of which applications are incorporated herein by reference.

### TECHNICAL FIELD

The present invention is related to novel use of piperine, and more specifically, it is related to a pharmaceutical or food composition for preventing and treating obesity comprising piperine or a salt thereof as an active ingredient.

### BACKGROUND ART

Obesity refers to a condition where fat tissues excessively accumulated within the body. In general, when their body mass index (BMI, an individual's body weight (kg) divided by the square of his or her height (m)) is greater than 25 (in Korea) or 30 (in the West), they are diagnosed as obesity. The obesity is caused by energy imbalance when over a long period, an excessive amount of nutrients with respect to the amount of energy consumption are ingested. The treatment of the obesity requires behavior therapy as well as reduction of repast and increase in the amount of exercise through improvement of life style.

In a dietary treatment, a reduction of caloric intake is most important, and thus it is recommended that caloric intake has to be less than ordinary caloric intake, by about 500 to 1000 kcal. It has been recently known that a weight reduction effect varies according to the composition of nutrition in a meal. An exercise is very important in that it is to prevent a reduced weight from increasing again. In order to achieve a treatment effect, an exercise for at least 30 minutes a day is required.

Besides the improvement of life style, a drug may be used for treatment of obesity. The drug used for the obesity treatment may be largely divided into an appetite suppressant and a fat absorption inhibitor. Also, there are two drugs allowed to be used over a long period, which are sibutramine as an appetite depressant and orlistat as a lipase inhibitor. Sibutramine may cause side effects such as headache, polydipsia (serious thirst), insomnia, and constipation. Furthermore, it may increase blood pressure and pulse frequency to some extent, and thus it is required to periodically check blood pressure and pulse frequency. Meanwhile, orlistat may cause side effects such as faces frequency, steatorrhea, and absorption reduction of fat soluble vitamin.

Meanwhile, in the modern society, the number of obesity patients is gradually increased according to nutrition improvement. Since the obesity increases the risk of diabetes mellitus, hyperlipidemia, sexual dysfunction, arthritis, cardiovascular disease, it has been continuously required to develop a novel drug for prevention and treatment of obesity.

Piperine is a compound found in long pepper, black pepper, white pepper, cubeb or dill, which has a structural formula of C₁₇H₁₉NO₃. Up to now, it has been known and reported that piperine has biological activities such as an antioxidant effect, an antimutagenic effect, and an anticancer effect. This plays a role of intensifying the bioavailability of drugs such as resveratrol. Through the treatment of an endothelial cell with piperine, an effective function for an immune response was achieved. In other words, adhesion of neutrophils and leukocyte is increased, and the movement of the p65 subunit of NK-kB from the cytoplasm to the nucleus is inhibited. For Swiss albino mice, one group was administered with benzo[α]pyrene (carcinogen causing lung cancer) in an amount of 50 mg/kg twice a week for 16 weeks while another group was administered with piperine in an amount of 100 mg/kg once a day for 4 weeks before administration of benzo[α]pyrene. As a result, the group pre-treated with piperine showed significantly high values of antioxidant indicators such as SOD (Superoxide dismutase), CAT (catalase), GPX (Glutathione peroxidase), GSH (Reduced glutathione), vitamin E, vitamin C or the like in the tissues. Thus, it was proven that piperine has an antioxidant effect.

Accordingly, piperine together with other plant extracts and phytochemicals has been commercially used for products of various dietary supplements, in the form of a composite, and has been on sale for the purpose of improvement of a general health function such as an antioxidant function.

The above information disclosed in this Technical Field and Background Art sections are only for enhancement of understanding of the background of the invention and therefore it may contain information that does not form the prior art that is already known in this country to a person of ordinary skill in the art.

### SUMMARY OF THE DISCLOSURE

Accordingly, the inventors of the present invention researched a new biological activity of piperine and then found that piperine inhibits the accumulation of visceral fat, and reduces the weight of a mouse model with intake of a high fat diet, thereby achieving a prevention/treatment effect of obesity. Then, they completed this invention by developing an obesity prevention/treatment composition including piperine or a salt thereof as an active ingredient.

Accordingly, an object of the present invention is to provide a novel use of piperine.

In order to accomplish this object, there is provided a pharmaceutical composition for preventing and treating obesity, which includes piperine or a pharmaceutically acceptable salt thereof as an active ingredient.

In accordance with another aspect of the present invention, there is provided a food composition for preventing and improving obesity, which includes piperine or a salt thereof as an active ingredient.

In accordance with a further aspect of the present invention, there is provided a use of piperine or a pharmaceutically acceptable salt thereof for preparing an agent for preventing and treating obesity.

In accordance with a still aspect of the present invention, there is provided a method for preparing an agent for preventing and treating obesity comprising administering an effective amount of piperine or a pharmaceutically acceptable salt thereof to a subject in need thereof.

As described above, the present disclosure relates to novel use of piperine, and provides a composition for preventing and treating obesity comprising piperine or a salt thereof as an active ingredient. The composition comprising piperine of the present disclosure or a salt thereof is useful for suppression of abdominal fat accumulation and body weight loss. Therefore, the composition can be effectively used to prevent and treat obesity.

### BRIEF DESCRIPTION OF THE DRAWINGS

FIG. 1 shows piperine's effect on adipocyte differentiation inhibition (A) and fat accumulation inhibition (B) in a 3T3L1 cell. A mark of *, ** or *** above a bar graph indicates that the result is significant in a range of *P<0.05, **P<0.01 or **P<0.001 in Student's t-test.

FIG. 2 shows a weight (A and B) and a weight gain (C) of a mouse with intake of an experimental diet [vertical axis unit: g (A, B), and g/10 weeks (C)][horizontal axis unit: day (A)]. Other marks above bar graphs indicate that the result is significant in a range of P<0.05 in one-way ANOVA and Duncan's multiple range tests.

FIG. 3 shows a diet intake (A) and a food efficiency ratio (B) of a mouse with intake of an experimental diet. Other marks above bar graphs indicate that the result is significant in a range of P<0.05 in one-way ANOVA and Duncan's multiple range tests. FER indicates a food efficiency ratio, which is a body weight gain (g) for the entire experimental raising period, divided by the diet intake (g) for the experimental raising period.

FIG. 4 shows respective parts' visceral fat weights per unit body weight in a mouse with intake of an experimental diet (epididymal fat, perirenal fat, mesenteric fat, and retroperitoneal fat). In the visceral fat tissue of the same part, other marks above bar graphs indicate that the result is significant in a range of P<0.05 in one-way ANOVA and Duncan's multiple range tests.

FIG. 5 shows photographs of visceral fat in respective parts of a mouse with intake of an experimental diet.

### DETAILED DESCRIPTION OF THE DISCLOSURE

Hereinafter reference will now be made in detail to various embodiments of the present invention, examples of which are illustrated in the accompanying drawings and described below. While the invention will be described in conjunction with exemplary embodiments, it will be understood that present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

The composition of the present disclosure comprises piperine represented by Chemical Formula 1 or a pharmaceutically acceptable salt thereof (or salt thereof) as an active ingredient and can be used to prevent and treat obesity.

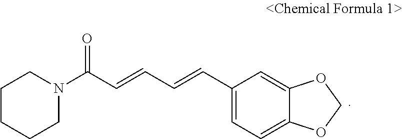

Piperine may be isolated and purified from natural materials or purchased commercially or prepared by chemical synthetic method which is well known in the art. Isolation and purification from natural materials may be performed from Long Pepper (Piper longum), Black Pepper (Piper nigrum), Cubeb (Piper ubeba) or Dill (Anethum graeolens) by solvent extraction method and chromatography which are well known in the art. For example, extraction of piperine from Long Pepper, Black Pepper, Cubeb or Dill may be prepared using water, C₁-C₆ alcohol such as ethanol, methanol, propanol, isopropanol, and butanol, organic solvent such as acetone, ether, chloroform, ethylacetate, methylenechloride, hexane, cyclohexane, petroliumether diethylether and benzene alone or in combination as well as above-mentioned solvent. In addition, isolation method by chromatography which is well known in the art, for example, by silicagel column chromatography, polar fraction may be prepared and the fraction could be isolated by high-performance liquid chromatography (HPLC).

In one example of the present invention, piperine's effect on differentiation and growth of adipocytes was determined. A mouse adipocyte line (3T3-L1) was differentiation-induced while being treated with piperine with different concentrations. Then, the extent of differentiation of adipocytes and the amount of intracellular fat were measured. As a result, it was found that the treatment of 3T3L1 with piperine significantly concentration-dependently reduced the differentiation of preadipocytes at a concentration of 10 μM or more, and concentration-dependently reduced the amount of intracellular fat (see Example 1).

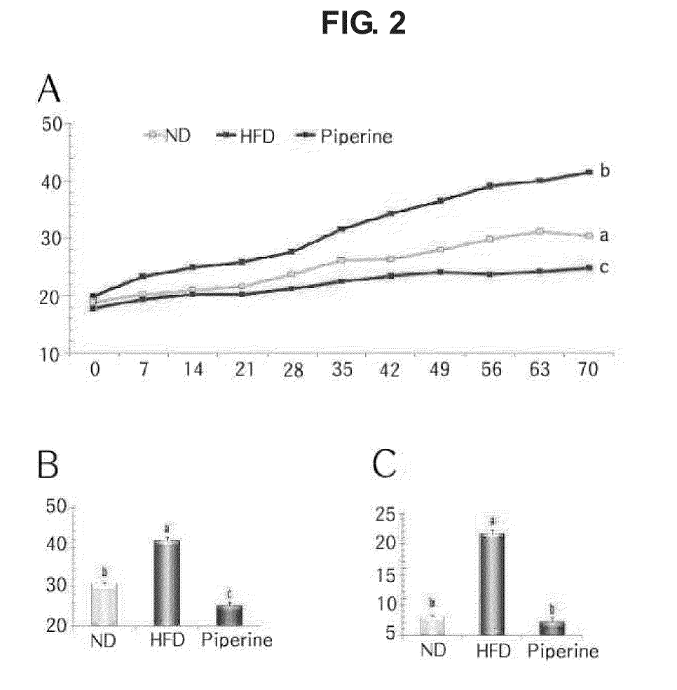

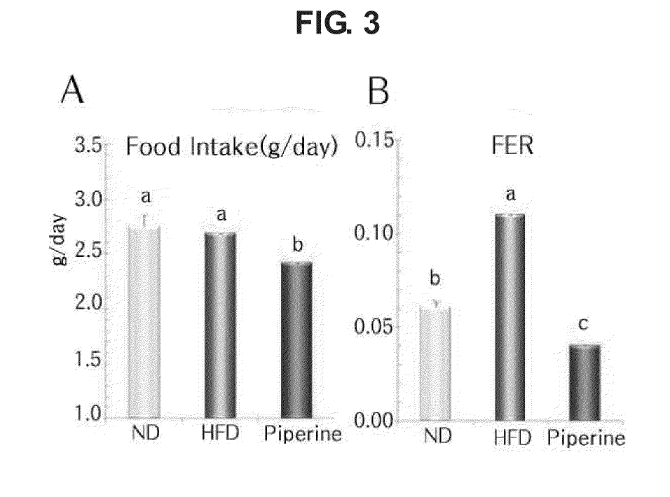

In another example of the present invention, for a mouse, piperine's effect on visceral fat accumulation and obesity induced by a high fat diet was determined. As a result, it was found that after intake of piperine-supplemented diet for 10 weeks, a final weight and an accumulated body weight gain were significantly decreased by 40% and 68%, respectively, (see FIG. 2), a daily diet intake was reduced (see FIG. 3), and a feed efficiency was reduced compared to the values for the control group (HFD). Furthermore, compared to a control group (HFD), the piperine-fed group, per unit body weight, showed a significant reduction of a fat weight (epididymal fat: 63%, perirenal fat: 85%, mesenteric fat: 68%, retroperitoneal fat: 63%, and a total weight of visceral fat including these four fats: 66%). Thus, it was determined that piperine has a very strong effect on a reduction in the amount of visceral fat. Accordingly, it can be found that in a high-fat diet mouse model, piperine can reduce visceral fat-pad weights, inhibit appetite, and reduces a body weight, thereby significantly treating or improving obesity (see Example 2).

Accordingly, the composition of the present disclosure comprising piperine or a pharmaceutically acceptable salt thereof as an active ingredient may be provided as a pharmaceutical composition for preventing or treating obesity. The composition of the present disclosure may comprise piperine or a pharmaceutically acceptable salt thereof in an amount of 0.001-99.999% (w/w) as well as an excipient as balance.

The expression “pharmaceutically acceptable” means that the salt is physiologically acceptable and normally causes no allergic or other similar adverse reactions when administered to human. The salt may be an acid addition salt formed from a pharmaceutically acceptable free acid. The free acid may be an organic acid or an inorganic acid. The organic acid includes citric acid, acetic acid, lactic acid, tartaric acid, maleic acid, fumaric acid, formic acid, propionic acid, oxalic acid; trifluoroacetic acid, benzoic acid, gluconic acid, methanesulfonic acid, glycolic acid, succinic acid, 4-toluenesulfonic acid, glutamic acid and aspartic acid, but is not limited thereto. And, the inorganic acid includes hydrochloric acid, bromic acid, sulfuric acid and phosphoric acid, but is not limited thereto.

The pharmaceutical composition according to the present disclosure may comprise a pharmaceutically effective amount of piperine or a salt thereof alone or in combination with one or more pharmaceutically acceptable excipient. The expression “pharmaceutically effective amount” refers to an amount required to exhibiting a desired effect as compared to a negative control group. Specifically, it refers to an amount sufficient to treat or prevent obesity. The pharmaceutically effective amount of piperine may be 0.01-100 mg/day/kg body weight. However, the pharmaceutically effective amount may vary depending on the particular disease and severity thereof, age, body weight, physical conditions and sex of the patient, administration route, treatment period, or other various factors.

The expression “pharmaceutically acceptable” means that the composition is physiologically acceptable and is nontoxic without causing allergic or other similar adverse reactions, such as gastroenteric trouble or dizziness, when administered to human. The composition of the present disclosure may be prepared into various formulations depending on administration routes along with the pharmaceutically acceptable excipient according to methods known in the art. The administration route includes oral and parenteral routes. Examples of the parenteral administration route include transdermal, intranasal, intraabdominal, intramuscular, subcutaneous and intravenous routes.

For oral administration, the pharmaceutical composition of the present disclosure may be formulated into powder, granule, tablet, pill, sugar-coated tablet, capsule, liquid, gel, syrup, suspension, wafer, or the like together with an adequate excipient for oral administration according to a method known in the art. The excipient may include a filler such as a sugar including lactose, dextrose, sucrose, sorbitol, mannitol, xylitol, erythritol, maltitol, etc., a starch including corn starch, wheat starch, rice starch, potato starch, etc., a cellulose including cellulose, methyl cellulose, sodium carboxymethyl cellulose, hydroxypropyl methylcellulose, etc., gelatin, polyvinylpyrrolidone, or the like. Also, a disintegrant such as crosslinked polyvinylpyrrolidone, agar, alginic acid, sodium alginate, etc. may be added. In addition, the pharmaceutical composition may further include an anticoagulant, lubricant, wetting agent, fragrance, emulsifier, antiseptic, or the like.

For parenteral administration, the pharmaceutical composition of the present disclosure may be formulated into injection, transdermal system or nasal inhaler together with an adequate excipient for oral administration according to a method known in the art. The injection should be sterilized and be protected from contamination by microorganisms such as bacteria and fungi. The injection may include a solvent or a suspension medium such as water, ethanol, polyol (e.g., glycerol, propylene glycol, polyethylene glycol, etc.), a mixture thereof and/or vegetable oil as an excipient. More specifically, the excipient may be Hank's solution, Ringer's solution, phosphate buffered saline (PBS) containing triethanolamine, sterile water for injection, or isotonic solution such as 10% ethanol, 40% propylene glycol and 5% dextrose. The injection may further include various antibacterial and antifungal agents such as paraben, chlorobutanol, phenol, sorbic acid, thimerosal, etc. for protection from contamination by microorganisms. Further, the injection may include an isotonic agent such as sugar or sodium chloride in most cases. The transdermal administration system may be in the form of ointment, cream, lotion, gel, topical solution, paste, liniment, aerosol, and so forth. As used herein, “transdermal administration” refers to a delivery of the effective amount of the active ingredient included in the pharmaceutical composition topically into the skin.

These formulations are described in the Remington's Pharmaceutical Science, 15th Edition, 1975, Mack Publishing Company, Easton, Pa., which is well known in the pharmaceutical chemistry field.

The inhaler may be in the form of a pressurized pack or an aerosol spray delivered from a nebulizer using an adequate propellant compound, e.g. dichlorofluoromethane, trichlorofluoromethane, dichlorotetrafluoroethane, carbon dioxide, etc. for easier delivery. A pressurized aerosol may be equipped with a valve for delivering a unit dosage. A gelatin capsule or cartridge used in the inhaler may include a powder mixture of lactose, starch or other matrix.

Other pharmaceutically acceptable excipients may be consulted from Remington's Pharmaceutical Sciences, 19th ed., Mack Publishing Company, Easton, Pa., 1995.

The pharmaceutical composition may be administered in combination with a compound known to have an effect of preventing and treating obesity.

The present disclosure provides a use of piperine or a pharmaceutically acceptable salt thereof for preparing an agent for preventing and treating obesity.

The present disclosure also provides a method for preventing and treating obesity comprising administering an effective amount of piperine or a pharmaceutically acceptable salt thereof to a subject in need thereof.

The piperine or a pharmaceutically acceptable salt thereof may be administered in an effective amount through various routes, including oral, transdermal, subcutaneous, intravenous or intramuscular routes. As used herein, the “effective amount” refers to an amount required to achieve a therapeutic effect for obesity including body weight loss when administered to a patient. The “subject” may be an animal, specifically a mammal including human. Also, it may refer to a cell, tissue or organ derived from an animal. The subject may be patient in need of treatment.

The piperine or a pharmaceutically acceptable salt thereof may be administered as it is or after being prepared into various formulations as described above. Specifically, they may be administered until the desired effect, i.e. the therapeutic effect for the metabolic disease selected from the group consisting of obesity, fatty liver, hyperlipidemia and diabetes, is achieved. Piperine or a pharmaceutically acceptable salt thereof may be administered via various routes according to methods known in the art. That is to say, they may be administered orally or parenterally, e.g. buccally, intramuscularly, intravenously, intradermally, intraarterially, intramedullarily, intradurally, intraabodominally, intranasally, intravaginally, rectally, sublingually or subcutaneously, or via gastrointestinal tracts, mucous membranes or respiratory organs.

Also, piperine of the present disclosure or a salt thereof may be provided in the form of a food composition for improving obesity. The food composition of the present disclosure may be in any form, including functional food, nutritional supplement, health food and food additives. The food composition may be prepared in various forms according to methods known in the art.

For example, piperine of the present disclosure may be prepared into tea, juice or drink or into granule, capsule or powder as health food. Also, piperine of the present disclosure may be mixed with a substance or active ingredient known to have an effect of improving a metabolic disease selected from the group consisting of obesity, fatty liver, hyperlipidemia and diabetes to prepare a composition.

And, as functional food, piperine of the present disclosure may be added to drinks (including alcoholic drinks), fruits and processed products thereof (e.g., canned fruit, bottled fruit, jam, marmalade), fish, meat and processed products thereof (e.g., ham, sausage, corned beef), confectionery and noodles (e.g., udon, buckwheat noodles, instant noodles, spaghetti, macaroni), fruit juice, drinks, cookies, yeot, dairy products (e.g., butter, cheese), vegetable oils, margarine, vegetable proteins, retort foods, frozen foods, seasonings (e.g., soybean paste, soy sauce, sauce), or the like.

The food composition of the present disclosure may preferably comprise the piperine or a salt thereof in amount of 0.01-50% (w/w) of the finally prepared food, but not limited thereto.

Also, piperine of the present disclosure may be prepared into powder or concentrate to be used as food additive.

The above-mentioned skills for nucleotides and proteins were referenced in Maniatis et al., Molecular Cloning: A Laboratory Manual, Cold Spring Harbor Laboratory, Cold Spring Harbor, N.Y. (1982); Sambrook et al., Molecular Cloning: A Laboratory Manual, 2d Ed., Cold Spring Harbor Laboratory Press (1989); Deutscher, M., Guide to Protein Purification Methods. Enzymology, vol. 182. Academic Press. Inc., San Diego, Calif. (1990)

### EXAMPLES

The examples and experiments will now be described. The following examples and experiments are for illustrative purposes only and not intended to limit the scope of this disclosure.

### Example 1

### Adipocyte Differentiation Inhibition Effect of Piperine

The effect of piperine on adipocyte differentiation and growth was investigated using mouse adipocytes (3T3-L1). Preadipocytes 3T3L1 were placed on a 12-well plate and cultured to confluency in a 5% CO₂ incubator at 37° C. using DMEM containing 1% penicillin-streptomycin, 1% non-essential amino acids and 10% fetal bovine serum (FBS). The cultured 3T3-L1 cells were differentiated into adipocytes by culturing for 2 days in a medium containing 0.5 mM methylisobutylxanthine/1 μM dexamethasone/1 μg/mL insulin (DMI). The cells were differentiated into mature adipocytes by further culturing for 2 days in DMEM containing 1 μg/mL insulin. Subsequently, they were further cultured for 10 days while changing DMEM every other day to obtain fully differentiated adipocytes.

Starting from the day when DMI was added to the 3T3-L1 cells, piperine was treated at concentrations of 0.1, 1, 10, 50 and 100 μM every other day. Piperine was purchased from Sigma and used after being dissolved in DMSO. For negative control, only DMSO was used. After the culturing for a total of 14 days, when the cells were fully differentiated, the medium was removed and the fats contained in the differentiated adipocytes were stained. For this, the cells were washed 2 times with phosphate buffered saline (PBS), immobilized in 10% buffered neutral formalin for 1 hour, washed again once with PBS, stained for 1 hour by adding 1 mL of Oil Red O, which specifically stains fats red, to the 12-well plate, and then washed 2 times with distilled water.

To measure the triglyceride level of the differentiated 3T3-L1 cells, the stained cells were dissolved in 1 mL of isobutanol and OD value was measured at 600 nm.

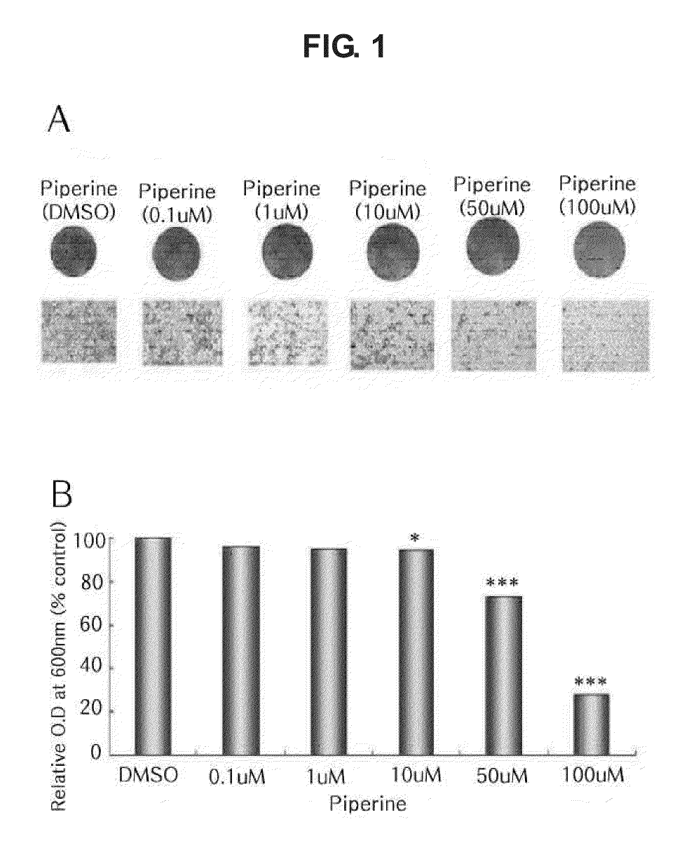

As seen from FIG. 1, piperine decreased the differentiation of the preadipocytes 3T3-L1 in a concentration-dependent manner at concentrations 10 μM or higher (A). Measurement of fats stained with Oil Red O also revealed the decrease in OD value in a concentration-dependent manner (B).

### Example 2

### Effect of Piperine on Reducing Body Weight and Suppression of Fat Accumulation

<2-1> Preparation of Test Diets and Rearing of Test Animals

The high-fat diet (HFD, 40% fat calorie, 17 g lard+3% corn oil/100 g diet) was used to induce obesity and piperine-containing diet had the same composition as the HFD except for containing 0.05% piperine. Normal diet (ND) was prepared according to AIN-76 rodent diet (see Table 1).

TABLE 1
Composition of test diets

| | ND | HFD | Piperine |
|---|---|---|---|
| | (g/kg diet) | (g/kg diet) | (g/kg diet) |
| Casein | 200 | 200 | 200 |
| D/L-Methionine | 3 | 3 | 3 |
| Corn starch | 150 | 111 | 110.5 |
| Sucrose | 500 | 370 | 370 |
| Cellulose | 50 | 50 | 50 |
| Corn oil | 50 | 30 | 30 |
| Lard | — | 170 | 170 |
| Vitamin complex | 10 | 12 | 12 |
| Mineral complex | 35 | 42 | 42 |
| Choline bitartrate | 2 | 2 | 2 |
| Cholesterol | — | 10 | 10 |
| tert-Butylhydroquinone | 0.01 | 0.04 | 0.04 |
| Pipeline | — | — | 0.50 |
| Total (g) | 1,000 | 1,000 | 1,000 |
| Fat (% calorie) | 11.5 | 39.0 | 39.0 |
| Total calorie (kJ/kg diet) | 16,439 | 19,315 | 19,315 |

5-week-old male C57BL/6J mice were accustomed to the laboratory environment for a week while feeding solid feed. They were randomly divided into the ND, HFD and carvacrol groups according to the randomized block design and reared for 10 weeks. Diet was provided between 10 and 11 a.m. every day along with water. Diet intake was measured every day, and body weight was measured every 3 days. In order to avoid transient body weight increase due to feed intake, body weight was measured 2 hours after removing the feedbox.

<2-2> Measurement of Body Weight, Efficiency of Feed and Abdominal Fat-Pad Weights

After feeding test diet for 10 weeks, final body weight and body weight gain was measured. As can be seen in FIG. 2, the piperine extract group showed significant decrease in body weight (40%) and body weight increase (68%) as compared to the high-fat diet control group.

As can be seen in FIG. 3, the amount of daily food intake during test period showed significant decrease in the piperine-fed group as compared to the high-fat diet control group and it showed effect on suppressing appetite. In addition, food efficiency ratio (FER) was calculated by dividing accumulated body weight gain during test period into total diet uptake from the first day of test diet to sacrifice day. As a result, the diet efficiency of the piperine-fed group was 64% reduced as compared to the high-fat diet control group and it showed effect of body weight loss of piperine was achieved from other mechanisms as well as suppressing appetite.

Further, regarding accumulation of abdominal fat, after fasting the test animal for at least 12 hours and anesthetizing with diethyl ether, blood, liver and abdominal fat tissues (epididymal, perirenal, mesenteric and retroperitoneal fat) were taken and weighed after washing with 0.1 M PBS (pH 7.4).

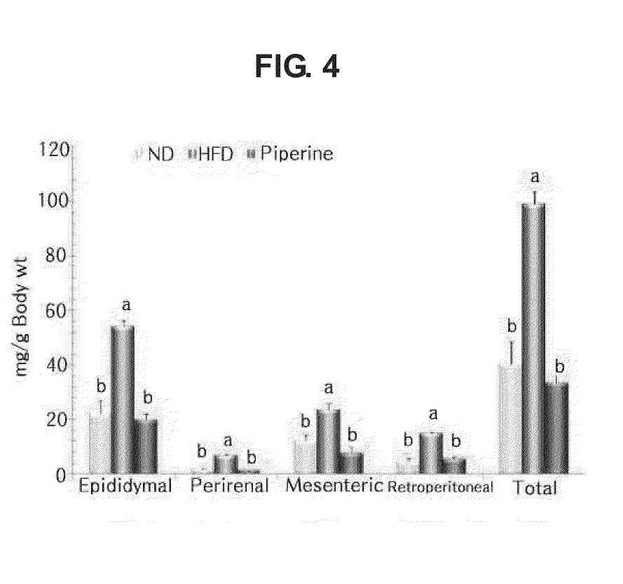

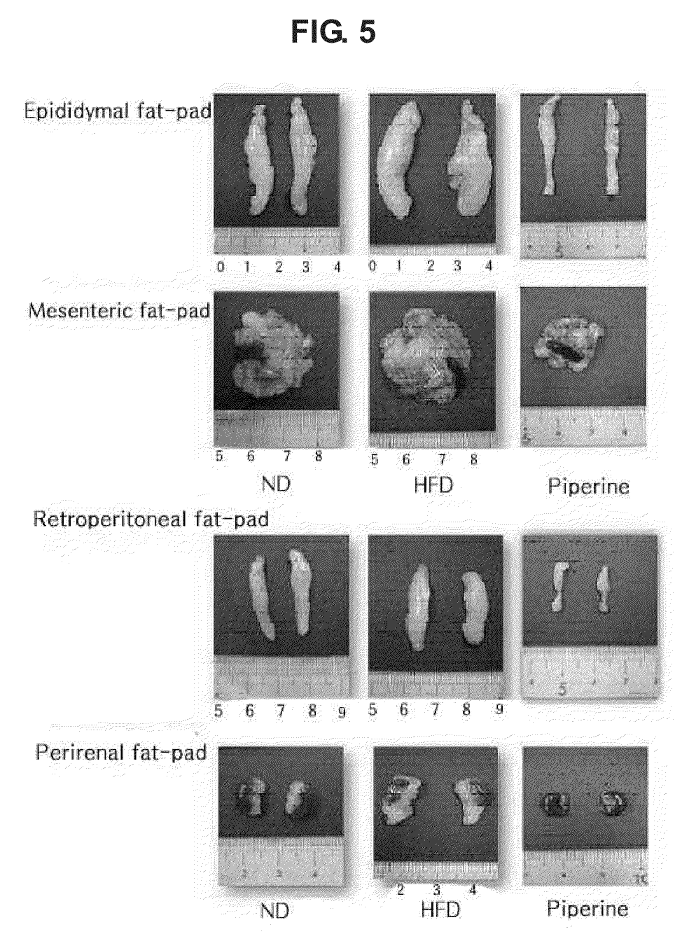

As results, as can be seen in FIGS. 4 and 5, after feeding test diet for 10 weeks, epididymal fat-pad weight, perirenal fat-pad weight, mesenteric fat-pad and retroperitoneal fat-pad weight were measured and epididymal fat-pad weight (63%), perirenal fat-pad weight (85%), mesenteric fat-pad weight (68%), retroperitoneal fat-pad weight (63%) and total abdominal fat-pad weights (66%) were significantly reduced as compared to the high-fat diet control group (p<0.001). Accordingly, it can be seen that piperine has an excellent effect of reducing body weight and abdominal fat-pad weights.

### Preparation Example 1

### Preparation of Powder

After mixing the below ingredients and filling in an airtight pouch and they were prepared into powder according to a well known method.

| | | |
|---|---|---|
| piperine | 50 | mg |
| crystallized cellulose | 2 | g |

### Preparation Example 2

### Preparation of Tablet I

After mixing the below ingredients and performing direct compression and they were prepared into tablet according to a well known method.

| | |
|---|---|
| piperine | 50 mg |
| crystallized cellulosed | 400 mg |
| magnesium stearate | 5 mg |

### Preparation Example 3

### Preparation of Tablet II

After mixing the below ingredients and performing direct compression and they were prepared into tablet according to a well known method.

| | |
|---|---|
| Piperine | 400 mg |
| crystallized cellulose | 100 mg |
| magnesium stearate | 5 mg |

### Preparation Example 4

### Preparation of Tablet III

Mixing Spiturina 55% (w/w), guagum enzyme extract 10% (w/w), Vitamin B₁ hydrochloride 0.01% (w/w), Vitamin B₆ hydrochloride 0.01% (w/w), DL-methionin 0.23% (w/w), magnesium stearate 0.7% (w/w), lactose 22.2% (w/w), corn starch 1.85% (w/w) and piperine 10% (w/w), then performing direct compression and they were prepared into tablet according to a well known method.

### Preparation Example 5

### Preparation of Capsule I

After mixing the below ingredients and filling into gelatin capsule and they were prepared into capsule according to a well known method.

| | |
|---|---|
| piperine | 30 mg |
| whey protein | 100 mg |
| crystallized cellulose | 400 mg |
| magnesium stearate | 6 mg |

### Preparation Example 6

### Preparation of Capsule II

After mixing the below ingredients and filling into gelatin capsule and they were prepared into capsule according to a well known method.

| | | |
|---|---|---|
| piperine | 300 | mg |
| corn starch | 100 | mg |
| crystallized cellulose | 100 | mg |
| magnesium stearate | 5 | mg |

### Preparation Example 7

### Injection

Active ingredient is dissolved into distilled water for injection according to a well known method, and adjust pH to 7.5 and the below ingredient were dissolved in distilled water for injection. Then filled in 2 ml of ampoule, sterilized and injection were prepared.

| | |
|---|---|
| piperine | 100 mg |

suitable amount of distilled water for injection

suitable amount of pH adjusting agent

### Preparation Example 8

### Seonsik

Alpharized and dried, by well known method, brown rice, barley, sweet rice, Job's tears were ground with granule size of 60 mesh. Black bean, black sesame and perilla were steamed and dried by well known method and ground with granule size of 60 mesh. The above prepared grains, seeds, and fucoxanthin extracts of <Example 1> were mixed with the ratio of below:

Grains: 30% (w/w) of brown rice, 15% (w/w) of Job's tears, 20% (w/w) of barley, 9% (w/w) of sweet rice;

Seeds: 7% (w/w) of perilla, 8% (w/w) of black bean, 8% (w/w) of black sesame;

3% (w/w) piperine, 0.5% (w/w) of Ganoderma lucidum, 0.5% (w/w) of Rehmannia glutinosa

### Preparation Example 9

### Chewing Gum

After mixing 20% (w/w) of gum base, 76.9% (w/w) of sugar, 1% (w/w) of flavor 2% (w/w) of water and 0.1% (w/w) of piperine, they were prepared into chewing gum according to a well known method.

### Preparation Example 10

### Chewing Gum

After mixing 60% (w/w) of sugar, 39.8% (w/w) of starch syrup, 0.1% (w/w) of flavor and piperine, they were prepared into candy according to a well known method.

### Preparation Example 11

### Biscuit

After mixing 25.59% (w/w) of first grade soft flour, 22.22% (w/w) of first grade plain flour, 4.80% (w/w) of refined sugar, 0.73% (w/w) of salt, 0.78% (w/w) of glucose, 11.78% (w/w) of palm shortening, 1.54% (w/w) of ammonium, 0.17% (w/w) of sodium bicarbonate, 0.16% (w/w) of sodium sulfite, 1.45% (w/w) rice powder, 0.0001% (w/w) of vitamin B₁, 0.0001% (w/w) of vitamin B₂, 0.04% (w/w) of milk flavor, 20.6998% (w/w) of water, 1.16% (w/w) of whole milk powder, 0.29% (w/w) of alternative milk powder, 0.03% (w/w) of calcium phosphate I, 0.29% (w/w) of sulfuric salt, 7.27% (w/w) of spray milk and 1% (w/w) of piperine, they were prepared into biscuit according to a well known method.

### Preparation Example 12

### Beverage

After mixing 0.26% (w/w) of honey, 0.0002% (w/w) of thiotic acid amide, 0.0004% (w/w) of nicotinic acid amide, 0.0001% (w/w) of riboflavin natrium HCl, 0.0001% (w/w) of pyridoxine HCl, 0.001% (w/w) inositol, 0.002% (w/w) ortic acid, 98.7362% (w/w) of water and 1% (w/w) of piperine, they were prepared into beverage according to a well known method.

As described, the composition comprising piperine of the present disclosure or a salt thereof is useful for suppression of abdominal fat accumulation and body weight loss. Therefore, the composition can be effectively used to prevent, improve and treat obesity.

What is claimed is:

1. A pharmaceutical composition for preventing and treating obesity comprising piperine represented by Chemical Formula 1 or a pharmaceutically acceptable salt thereof as an active ingredient:

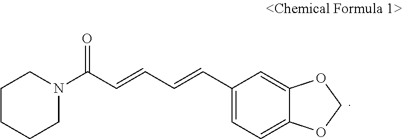

2. A food composition for preventing and improving obesity comprising piperine or a pharmaceutically acceptable salt thereof as an active ingredient.

3. A use of piperine or a pharmaceutically acceptable salt thereof for preparing an agent for preventing and treating obesity.

4. A method for preparing an agent for preventing and treating obesity comprising administering an effective amount of piperine or a pharmaceutically acceptable salt thereof to a subject in need thereof.
